(19) United States
(12) Patent Application Publication
(10) Pub. No.: US 2005/0060175 A1
(43) Pub. Date: Mar. 17, 2005

(54) System and method for comparing candidate responses to interview questions

(75) Inventors: Michael Allen Farber, Moorestown, NJ (US); Hal Marc Cohen, Cherry Hill, NJ (US)

Correspondence Address: Daniel H. Golub, 1701 Market Street, Philadelphia, PA, 19103, US

(73) Assignee: Trend Integration , LLC, Cherry Hill, NJ (US)

(21) Appl. No.: 10/896,525

(22) Filed: Jul. 22, 2004

Related U.S. Application Data
(60) Provisional application No. 60/502,307, filed on Sep. 11, 2003.

(51) Int. Cl. G06F 17/60; G09B 19/18; G09B 3/00; G09B 7/00; A61B 5/04

(52) U.S. Cl. 705/1; 434/322; 434/107

(57) ABSTRACT

A system and method for interviewing candidates and comparing candidate responses to interview questions includes an interactive voice response unit that automatically interviews the first and second candidates by sequentially prompting each candidate with the stored interview questions; and stores verbal responses of the candidates in a database.

### CROSS-REFERENCE TO RELATED APPLICATION

The present application claims priority to U.S. Provisional Patent Application No. 60/502,307, filed Sep. 11, 2003, entitled “Data Collection, Retrieval and Analysis Process,” incorporated herein in its entirety by reference.

### FIELD OF THE INVENTION

The present application relates generally to systems and methods for automatically interviewing candidates and, more specifically, to systems and methods for reviewing candidate responses received during automated interviews.

### BACKGROUND OF THE INVENTION

The widespread use of the Internet has enabled the rapid collection and exchange of many different types of information that can be delivered in a variety of mediums. Therefore a real need exists for a system that allows the user to leverage the power of this communication technology in the context of collection and analysis of information required for many different business and personal decision-making processes. The purpose of the present invention is to provide the user with a flexible, efficient system for the timely collection and analysis of information required to make a variety of important business and/or personal decisions. Examples of the diverse environments in which the system can be applied include but are not limited to: job applicant interviewing and hiring; college applicant interviewing and acceptance; collection, reporting and analysis of information in the context of personal dating services; and collection, reporting and analysis of information from candidates for political office.

IVR systems for automatically conducting job interviews exist in the prior art. However, such systems lack an efficient and effective means for comparing verbal responses recorded by such systems during the automated interview process. The present invention addresses this shortcoming in existing automated interview systems.

### SUMMARY OF THE INVENTION

The present application is directed to a computer-implemented system and method for interviewing at least first and second candidates and comparing candidate responses to interview questions. A plurality of interview questions for cueing the candidates is stored in a database, wherein the plurality of interview questions includes at least first and second questions. An interactive voice response unit automatically interviews the first candidate by sequentially prompting the first candidate with each of the plurality of stored interview questions; and storing a verbal response (i.e., an audible narrative response) of the first candidate to the first interview question in the database and storing a verbal response of the first candidate to the second question in the database. The interactive voice response unit also automatically interviews the second candidate by sequentially prompting the second candidate with each of the plurality of stored interview questions; and storing a verbal response of the second candidate to the first interview question in the database and storing a verbal response of the second candidate to the second question in the database.

An interface, operable after completion of the automatic interviewing of the first and second candidates by the interactive voice response unit, is then used for selecting, from the database, the stored verbal response of the first candidate to the first interview question and the stored verbal response of the second candidate to the first interview question. A processor, responsive to the interface, sequentially plays the stored verbal response of the first candidate to the first interview question and the stored verbal response of the second candidate to the first interview question without playing any other stored interview question response between the playing of the stored verbal response of the first candidate to the first interview question and the stored verbal response of the second candidate to the first interview question. By facilitating the sequential comparison of verbal responses from different candidates to the same interview question, the present invention provides an effective and efficient means for comparing candidate responses received during the automated interview process.

In one embodiment, the stored verbal response of the first candidate to the first interview question and the stored verbal response of the second candidate to the first interview question are selected for review using a graphical user interface. In a more specific embodiment, the graphical user interface contains first, second, third and fourth response areas arranged in a grid pattern, wherein the first response area corresponds to the stored verbal response of the first candidate to the first interview question, the second response area corresponds to the stored verbal response of the first candidate to the second interview question, the third response area corresponds to the stored verbal response of the second candidate to the first interview question, and the fourth response area corresponds to the stored verbal response of the second candidate to the second interview question.

In accordance with a further aspect, the present invention is directed to a system and method for generating a plurality of different sequences of interview questions for conducting automated interviews of candidates, wherein each of the different sequences of interview questions corresponds to one or more of a plurality of different positions associated with the automated interviews. A database is provided that stores a plurality of different interview questions. A graphical-user interface, coupled to the database, displays a plurality of labels each of which represents one of the different interview questions.

The graphical user-interface also includes an assembly area, displayed simultaneously with the labels, for assembling each of the sequences of interview questions. The graphical user interface includes functionality operable by a user for: (i) assembling a first sequence of interview questions corresponding to a first position by associating a first plurality of the labels with the assembly area; (ii) associating the first sequence of questions with the first position; (iii) assembling a second sequence of interview questions corresponding to a second position by associating a second plurality of the labels with the assembly area; and (iv) associating the second sequence of questions with the second position. The first sequence of questions is different from the second sequence of questions.

The graphical user interface also includes functionality that streamlines the building of interview question sequences by facilitating the reuse of interview questions in multiple interview question sequences. For example, once a question such as “Please describe your salary requirements?” is recorded and stored in the database (and represented as a label on the graphical user interface), a user building a sequence of interview questions for one position (e.g., a secretarial position) can initially select the interview question for inclusion in the sequence of questions to be used for conducting automated interviews for the secretarial position and then later, when building a sequence of interview questions for a further position (e.g., a custodial position) the user can again select the same interview question for inclusion in the sequence of questions to be used for conducting automated interviews for the custodial position.

Thus, once an interview question is recorded and stored in the database, the interview question can be used to build interview question sequences for different positions without re-recording of the question. In accordance with this aspect of the invention, the graphical user interface includes functionality operable by the user for associating at least one label representing a common question with the assembly area during assembly of the first sequence and for associating the at least one label representing the common question with the assembly area during assembly of the second sequence. An interactive voice response unit, coupled to the database, automatically interviews candidates for the first position using the first sequence of interview questions assembled using the graphical user interface and automatically interviews candidates for the second position using the second sequence of interview questions assembled using the graphical user interface.

In a preferred embodiment, the plurality of labels displayed on the graphical user interface comprise a plurality of icons each of which represents one of the different interview questions, the user assembles the first sequence of interview questions corresponding to the first position by dragging and dropping a first plurality of the icons into the assembly area, and the user assembles the second sequence of interview questions corresponding to the second position by dragging and dropping a second plurality of the icons into the assembly area. Each of the icons optionally corresponds to a narrative interview question stored in an audible format in the database. In addition, the graphical user interface optionally includes functionality that allows the user to add one or more interview questions customized for a specific candidate for the first position to the first sequence of interview questions prior to the automated interview of the specific candidate for the first position. The graphical user interface also optionally includes functionality for associating one or more attributes with an interview question stored in the database.

In accordance with a still further aspect, the present invention is directed to a system and method for generating at least one interview question for conducting an automated interview of a candidate for a position. A server receives a request submitted by a user to create an interview question, prompts the user to input a label to be assigned to the interview question and receives the label from the user. At least one interactive voice response unit, coupled to the server, conducts a telephone call with the user, established after receipt of the request, wherein the at least one interactive voice response unit prompts the user to speak the interview question during the telephone call. A database, coupled to the server and the at least one interactive voice response unit, stores a recording of the interview question spoken by the user during the telephone call, and the at least one interactive voice response unit later conducts an automated interview of the candidate for the position by at least playing the stored question for the candidate during the automated interview.

In a preferred embodiment, the server also prompts the user to input a telephone number associated with the user, receives the telephone number from the user, and automatically initiates the telephone call to the user using the telephone number received from the user. The server also optionally prompts the user to input a time duration time associated with a response to the interview question. Finally, in the preferred embodiment, the server provides the user with an option to listen to the recorded interview question, and an option to rerecord the interview question.

### BRIEF DESCRIPTION OF THE DRAWINGS

FIG. 1 depicts a graphical user-interface for entering information about a position that will be the subject of automated interviews, in accordance with the present invention.

FIG. 2 depicts a graphical user-interface for entering information about candidates that will be interviewed, in accordance with the present invention.

FIG. 3 depicts a graphical user-interface for displaying information about positions that will be the subject of automated interviews, in accordance with the present invention.

FIG. 4 depicts a graphical user-interface for assembling a set of stored interview questions for a particular position that is the subject of automated interviews, in accordance with the present invention.

FIG. 5 depicts a graphical user-interface for assigning attributes to interview questions, in accordance with the present invention.

FIG. 6 depicts a graphical user-interface for assigning candidate specific questions to a candidate, in accordance with the present invention.

FIG. 7 depicts a graphical user-interface for adding/modifying candidate information, in accordance with the present invention.

FIG. 8 depicts a graphical user-interface for reviewing candidate information, in accordance with the present invention.

FIG. 9 depicts a graphical user-interface for selectively reviewing verbal responses of candidates, in accordance with the present invention.

FIG. 10 depicts a further example of the graphical user-interface shown in FIG. 9.

FIG. 11 depicts a further example of the graphical user-interface shown in FIG. 9, wherein the user is provided with an ability to record a rank for each interview response using the interface.

FIG. 12 illustrates a system for implementing the functionality illustrated in FIGS. 1-11.

### DETAILED DESCRIPTION OF THE PREFERRED EMBODIMENT

The system present invention architecture works equally well for many types of data collection, retrieval and analysis processes. Examples of the diverse environments in which the system can be applied include but are not limited to: job applicant interviewing and hiring; college applicant interviewing and acceptance; collection, reporting and analysis of information in the context of personal dating services; and collection, reporting and analysis of information from candidates for political office. The following describes a detailed example of the present invention as applied to automated job applicant interviewing.

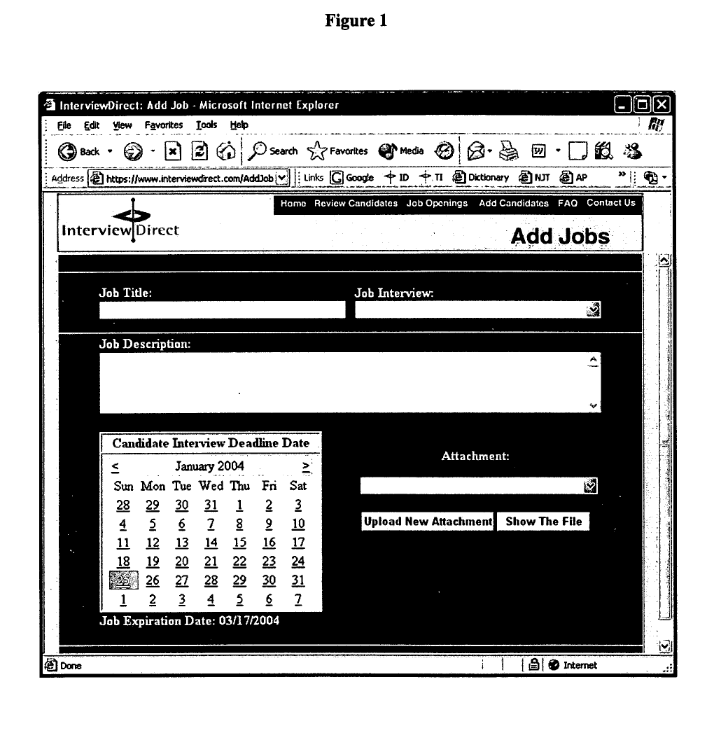

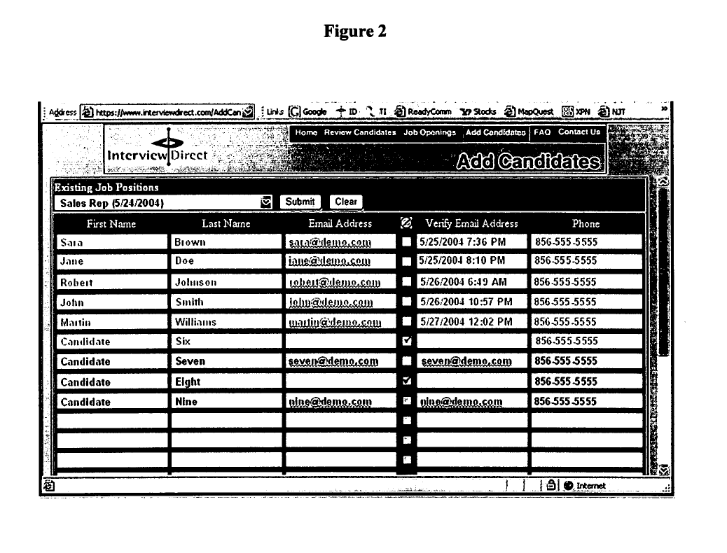

The type of data or information collected by the system is dependent upon the specific application. In the use of the system as applied to automated job applicant interviewing, the User, or employer/recruiter in this case, collects information pertaining to the background and experience of a certain job applicant (or candidate), for example “information related to John Smith”. In this embodiment, the system optionally creates and associate a unique PIN with “information related to John Smith”, and as described more fully in connection with FIGS. 1 and 2 below, the User selects a designation (“Job Position”) such as “Sales Rep Position” that information related to each job applicant for the Sales Rep position is to be associated.

The User selects or creates a series of Interview Questions (“Job Interview”) that the system associates with the information related to John Smith via the PIN associated with such information by the system. The Job Interview could consist of a sequence of audible questions administered over the telephone using an interactive voice response (“IVR”) system. The User may also select a date after which time the PIN provided to the job applicant becomes deactivated and by doing so denies the job applicant access to the Job Interview (“Interview Deadline Date”).

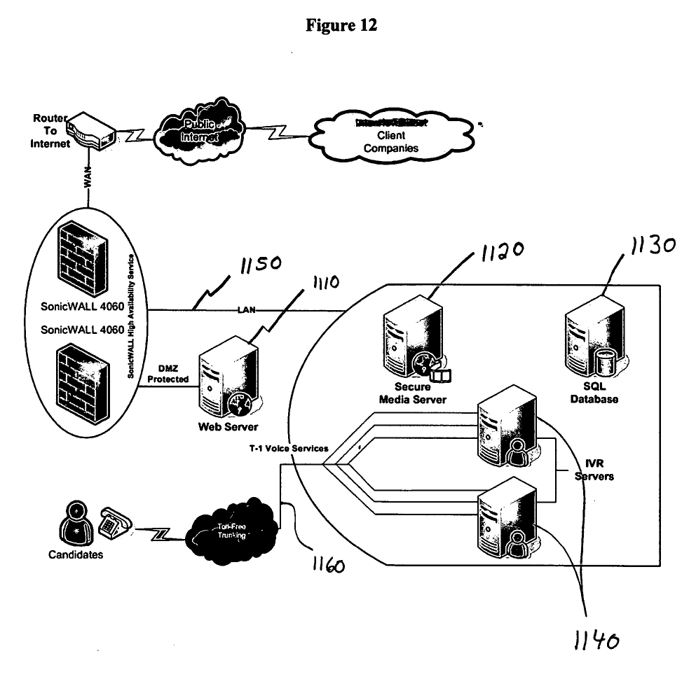

A computer (e.g., server 1110 shown in FIG. 12) coupled to one or databases (e.g., secure media server 1120 and SQL database 1130 shown in FIG. 12) linked with an IVR (e.g., IVR servers 1140) is configured to: (i) allow the User to create and store Job Interviews; (ii) allow the User to create and store a job interview question in a variety of formats (such as graphical text, audio, graphical, video and/or visual) either created by or at the direction of the User or provided to the User by the system (“Interview Question”); (iii) allow the User to designate groups or banks in which an Interview Question could be stored such as “Sales Rep”, “Administrator”, “Project Manager”, “General” etc. (“Interview Question Bank”); (iv) allow the User to add, modify and delete an Interview Question in each such Interview Question Bank; (v) allow the User, for identification purposes, to apply a designation, to an Interview Question contained within such Question Bank (“Question Label”) such as “Educational Background” for the question, “What is your educational background? or “Why Qualified” for the questions, “Why do you think you are qualified for this position?” etc.; (vi) allow the User to create, store and designate an Interview Question to be included only in the Job Interview transmitted to a specific job applicant selected by the User (“Applicant Specific Question”); (vii) allow the User to create a Job Interview by selecting Interview Questions from a single or different Question Banks and determining the order in which such selected Interview Questions will be transmitted; (iix) allow the User to save, modify and delete each such Job Interview created by the User; (ix) allow the User to designate groups or banks in which such Job Interviews created by the User could be stored (“Job Interview Bank”) such as “Sales Rep Interviews”, “Administrator Interviews”, “Project Manager Interviews”, “General Screen Interviews” etc.; (x) allow the User, for identification purposes, to apply a designation, to each such Job Interview created by the User contained within such Job Interview Bank (“Job Interview Label”) such as “Administrator Interview—HR Dept.”, “Project Manager Interview—IT Dept.”, “General Screen Interview—54” etc.; (ix) associate a Job Interview with a User and/or different Users; (xii) associate an Interview Question with a User and/or different Users; (xiii) associate Job Interview Banks with a User and/or different Users; (xiv) associate Interview Question Banks with a User or different Users; and (xv) enable the User or Users to review each Job Interview and Interview Question associated with such User or Users by outputting, automatically or upon such User or Users request, in a format predefined by the system or selected by the User or Users. Such format could include: (1) graphical text—either Interview Questions input by or at the direction of the User via computer keystroke operation or by way of application of speech to text technology as applied to Interview Questions input by or at the direction of the User via audio recording spoken by or at the direction of the User; (2) audio—either playback of Interview Questions input by or at the direction of the User via audio recording spoken by or at the direction of the User or by way of application of text to speech technology to Interview Questions input by or at the direction of the User via computer keystroke operation; (3) video—playback of Interview Questions input by or at the direction of the User via video recording device; (4) graphical; (5) visual; and (6) any combination of formats set forth in subparagraphs 1, 2, 3, 4 and 5 above.

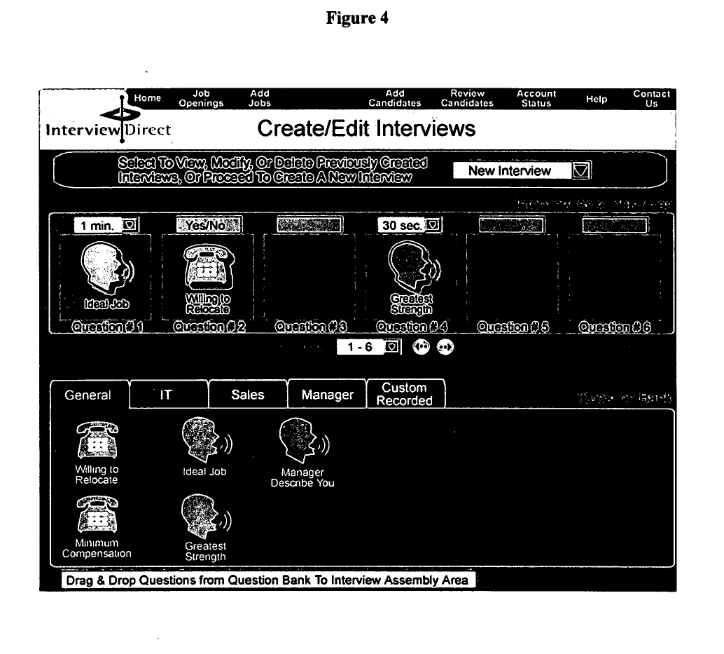

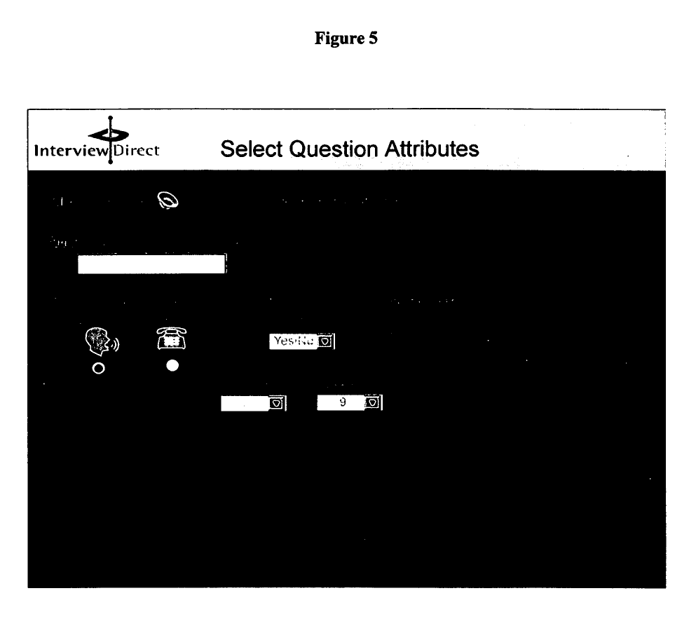

The system is configured to: (i) create and/or designate a different, unique graphical and/or textual representation that the system associates with an Interview Question associated with such User (“Interview Question Icon”); (ii) output each such Interview Question Icon in graphical, visual format over a computer network (“Job Interview Assembly Screen”); (iii) output each such Interview Question Icon arranged in a visual format on such Job Interview Assembly Screen so as to indicate the Question Label and Job Interview Bank to which the Interview Question represented by such Interview Question Icon is associated; (iv) enable the User to create a Job Interview and associate such Job Interview with such User by: (a) accessing the Job Interview Assembly Screen; (b) selecting which of such Interview Questions to include in the Job Interview using a computer point and click operation to select the Question Icons associated with Interview Questions to be included in the Job Interview (“Point and Click Interview Question Selection”); (c) upon such Point and Click Interview Question Selection to select the order in which each such selected Interview Question will be transmitted (this could be performed by the User via a drop down menu or dialog box displayed after each such Point and Click Interview Question Selection and/or after all such Point and Click Interview Question Selections have been performed); (d) upon such Point and Click Interview Question Selection to select (such selection could be performed by the User via a drop down menu or dialog box displayed after each such Point and Click Interview Question Selection and/or after all such Point and Click Interview Question Selections have been performed) other criteria associated with each such selected Interview Question (“Interview Question Characteristics”) such as: (1) the type of response required by the Interview Question (such as verbal, audio, telephone key punch, video, graphical text and/or any combination thereof), (2) the duration time permitted for the response to such Interview Question, (3) in the event a key punch response is required by the Interview Question; the type of telephone key punch response required (yes/no, numeric, multiple choice), the valid keys that may be used to respond, and the total number of key punch inputs required to respond, (4) the medium of transmission and format (such as graphical text, audio, graphical, video and/or visual) by which the Interview Question is to be transmitted to the Job Applicant by the system (it is not required that the Interview Question input format be the same as the format transmitted to the job applicant by the system as the system could utilize various format conversion tools such as voice to text and speech to text technologies); and (e) upon such Point and Click Interview Question Selection providing for Job Interview and Interview Question User Review Capability. See FIGS. 4 and 5 discussed below.

The system could is also configured to: (i) include a specially designated graphical area in the Job Interview Assembly Screen (“Job Interview Assembly Area”) into which the User may place, using a computer mouse drag and drop operation, the Question Icons associated with Interview Questions to be included in the Job Interview (“Drag and Drop Interview Question Selection”); (ii) provide a design and layout of the Job Interview Assembly Area so that the location within said area in which the User selects to makes such Drag and Drop Interview Question Selection corresponds to the order in which each selected Interview Question will be transmitted to the job applicant by the system; (iii) provide a design and layout of the Job Interview Assembly Area that includes a designated area which the user may select the Interview Question Characteristics of each Interview Question associated with each such Drag and Drop Interview Question Selection (such selection could be performed by the User via a drop down menu or dialog box displayed after each such Drag and Drop Interview Question Selection and/or after all such Drag and Drop Interview Question Selection have been performed) and other criteria associated with each such selected Interview Question; and (iv) upon such Drag and Drop Interview Question Selection provide for Job Interview and Interview Question User Review Capability. See FIGS. 4 and 5.

As discussed more in connection with FIG. 5 below, the system includes the following process to enable the User to create a job interview question: (i) the User inputs a request to server 1110 indicating the desire to create a question; (ii) the server prompts the User to input the Question Label to be assigned to such recorded question; (iii) the server prompts the User to input the Interview Question Bank to which such recorded question will be assigned; (iv) the server prompts the User to input the type of response (verbal, audio, telephone key punch, video, graphical text and/or any combination thereof) required by the question; (v) the server prompts the User to input to the system the duration time allotted for the response to such question; (vi) in the event a key punch response is required by the question, the server prompts the User to input the type of telephone key punch response (yes/no, numeric, multiple choice), the valid keys that may be used to respond, and the total number of key punch inputs required to respond; (vii) the server prompts the User to input to the system the User's telephone number at the User's location; (viii) the server upon receipt of such input of the User's telephone number, causes IVR 1140 to initiate a telephone call to the User at such telephone number; (ix) upon establishing such telephone communication link between IVR 1140 and the User, IVR 1140 prompts the User to record the question; (x) upon such prompt by the system, the User speaks the question to be recorded, (xi) the system records the spoken question and provides the User with the ability to listen to the question as recorded, rerecord the question and submit the recording of the question to the system for storage by the system, once the User is satisfied with the question as recorded; (xii) the system records and stores the spoken question in a format that can be played back and listened to by the job applicant; (xiii) the system stores the recorded question for use in a specific or different interviews by the User; (xiv) upon the User's election to store the recorded question, the system saves the recorded question in such a way so that all the response attributes selected by the User in subparagraphs (ii) through (vi) above are associated with the recorded question by the system; and (xv) upon the User's election to store the recorded question, the User optionally elects to have the recorded question converted to text using speech recognition technology and saved by the system in textual format.

As shown in FIG. 2, the User inputs into the system, name, e-mail address and phone number of the job applicant, John Smith; the individual from whom the information about John Smith is to be collected by the system. The job applicant, John Smith is contacted automatically by the system by e-mail or phone and is provided with some or all of the following information: (i) a request to provide information about John Smith; (ii) a toll free number to call to access the system to provide such information; (iii) the PIN associated with such information; (iv) that upon calling the toll free number there will be a prompt to enter the PIN via applicant's telephone keypad; (vii) other instructions for providing the requested information; and (ii) any other information the User selects to accompany such request such as the job title, job description, and the Interview Deadline Date (collectively referred to as the “Job Interview Invitation”).

The job applicant, John Smith, upon calling the toll free number to access the system and entering the PIN provided to him hears each question comprising the Job Interview, and following the transmission of each question is prompted by the system to and given an opportunity to provide an answer to such question via an audible response (e.g., a narrative response in the case of a non-multiple choice question) or telephone keypad response and each such response would be transmitted to and recorded by the system (“Job Interview Responses”).

The system is configured so that upon the system's receipt of the job applicant's PIN, the system transmits a question or series of questions based on the job applicant's response or responses. For example, if the job applicant responds yes to the question, “Are you willing to relocate?”, the system could be preprogrammed to ask the job applicant to choose from two different job locations by pressing either 1 for the Atlanta location or press 2 for the Detroit location. The system could be configured to apply such a methodology in multiple layers during the interview process. In the current example, if the job applicant pressed 1 for the Atlanta location, the system could be preprogrammed to ask the job applicant questions specific to the job at the Atlanta location.

The system may also be configured so that prior to the job applicant responding to the Job Interview, the job applicant is prompted to and/or required to indicate consent to have the Job Interview Responses recorded and/or stored by the system (“Job Applicant Consent”). The system could be configured so that the job applicant, following a prompt transmitted to the job applicant, could perform such Job Applicant Consent via telephone keypunch (ie. “Press 1 if you consent to have your responses recorded or press 2 to indicate you do not consent to have your responses recorded”). The system could be configured, using speech recognition technology, so that the job applicant, following a prompt transmitted to the job applicant, could perform such Job Applicant Consent by speaking a certain word or phrase into the telephone receiver (ie. “At the tone say yes if you consent to have your responses recorded or say no to indicate you do not consent to have your responses recorded”).

In a preferred embodiment, the IVR system is configured to automatically interview multiple candidates for each position, and to receive and store the Job Interview Response of each job applicant interviewed for the position by the IVR system. During the interview process, the IVR system automatically prompts each of the candidates with a common set of interview questions (as well as, in some cases, candidate specific questions), and records the candidates' responses, which will often include audible narrative responses to various questions from the common set of interview questions.

A computer containing a computer database linked with the IVR system is configured to: (i) create, receive, store and transmit the PIN associated with the information about each job applicant; (ii) associate the PIN with information about a job applicant; (iii) create, receive, store and transmit the Job Interview associated with the information about a job applicant; (iv) allow the User to select, store and create the Job Interview to be associated with the information about a job applicant; (v) receive, store and transmit the Job Interview Responses of each job applicant.

The IVR system is linked with a database and a server (such as a web server) that delivers to the User, over a LAN and/or WAN, via an Intranet, the Internet, or other distributed network, in any of a variety of formats such as visual, graphical, textual, video and audio: (i) information about each job applicant; (ii) the PIN associated with the information about each job applicant in a manner wherein such association is perceptible to the viewer; (iii) the Job Interview associated with the information about each job applicant in a manner wherein such association is perceptible to the viewer; (iv) the PIN associated with each job applicant in a manner wherein such association is perceptible to the viewer; (v) the Job Interview Responses associated with the job applicants associated with the information about each such job applicant wherein each such association is perceptible to the viewer; (vi) a list of the job applicants that were sent a Job Interview Invitation that may include the date each such Job Interview Invitation was transmitted; (vii) an aggregate summary of the number of the job applicants that were sent a Job Interview Invitation that may include the date each such Job Interview Invitation was transmitted, such summary may also include the Job Position to which each such job applicants are associated in a manner wherein each such association is perceptible to the viewer; (viii) the Interview Deadline Date associated with each job applicant in a manner wherein each such association is perceptible to the viewer; and (ix) an aggregate summary of the number of job applicants that provided Job Interview Responses associated with a Job Position in a manner wherein each such association is perceptible to the viewer.

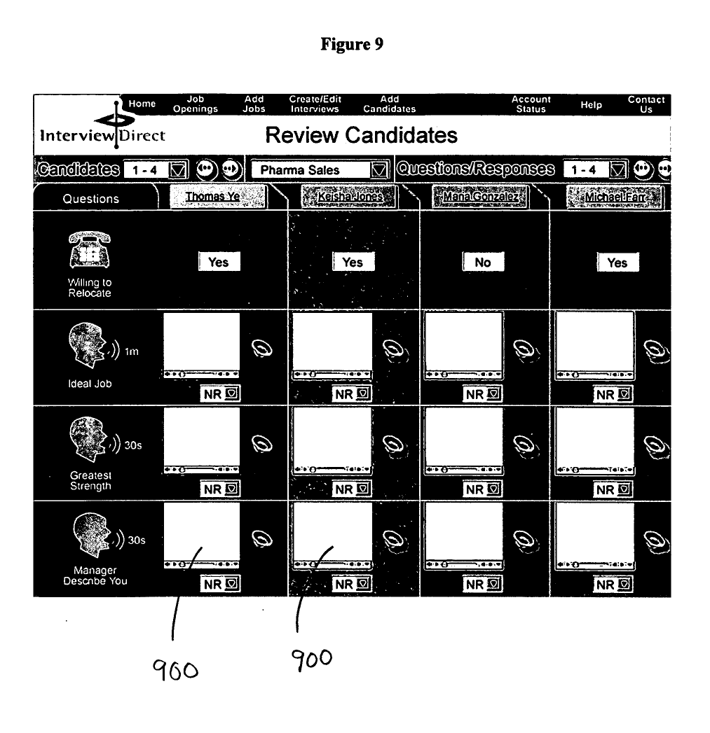

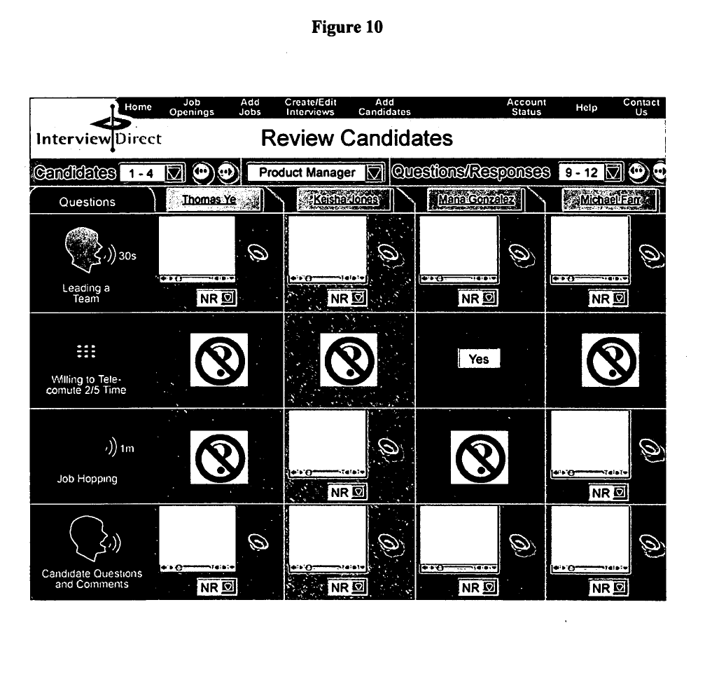

In one embodiment, the system is configured to display the data output in a interactive visual display format in which: (i) a grid is displayed in graphical format; (ii) each cell within such grid (with the exception of the First Cell) located in the uppermost horizontal row of such grid contains, in a visual, video, graphical and/or textual and/or audible format (“Variable Data Medium Format” or “VDMF”), a designation of specific information about a job applicant such as “John Smith” and/or the PIN associated with such information (“Job Applicant Heading”); (iii) each cell comprising the column of cells below each such Job Applicant Heading (“Applicant Response Location” or “ARL”) contains a representation, in VDMF, of the data provided by the job applicant associated with such Job Applicant Heading (for example, each ARL below the “John Smith” Job Applicant Heading would contain data collected from John Smith) in response to the specific Interview Question represented in the leftmost cell of the row of such grid wherein such ARL is located (“Response Data Representation” or “RDR”); (iv) the leftmost cell located in the uppermost horizontal row, First Cell, of such grid contains a representation, in VDMF, of a Job Interview associated with the information about each job applicant designated by each such Job Applicant Heading represented in the remaining cells of such uppermost horizontal row of such grid (“Job Interview Heading”); and (v) each cell comprising the column of cells below such Job Interview Heading (“Interview Question Location” or “IQL”) contains a representation, in VDMF, of a specific Interview Question comprising the Job Interview represented by such Job Interview Heading (“Interview Question Representation” or “IQR”). See discussion of FIGS. 9 and 10 below.

An icon may be displayed within each ARL (“Data Delivery Icon” or “DDI”) where upon the User's selection of such DDI (such selection may be performed via computer mouse point and click operation, computer mouse rollover operation, computer touch screen operation, computer voice recognition command operation, and/or computer visual command recognition) the job applicant's response represented in such ARL could be delivered to the User in a variety of formats such as audio, video, textual, graphical, and/or visual formats (“ARL Data Delivery”).

For example, if the User selected the DDI located within the ARL under the “John Smith” Job Applicant Heading located in the row wherein the IQL contained in such row represented the Interview Question, “What is your desired salary?”, John Smith's recorded response to this question would be played back for the User to review (assuming the response provided to the system was in an audio format or was converted to audio format by the system). This aspect of the invention is discussed further in connection with FIGS. 9 and 10 below. Among other things, this aspect of the invention allows the User to select and sequentially play back each candidate's recorded verbal response to a given Question, i.e., “What is your desired salary?” without playing back in between any verbal responses of the candidates to other Questions from the Interview. By allowing the User to juxtapose in time verbal responses from multiple candidates to the same Question, the present invention facilitates and streamlines the efficient and effective comparison of candidate responses received during the automated interviewing process.

In one embodiment, (i) a DDI is displayed within each IQL; and (ii) where upon the User's selection of such DDI (such selection may be performed via computer mouse point and click operation, computer mouse rollover operation, computer touch screen operation, computer voice recognition command operation, and/or computer visual command recognition), the data represented in such IQL is delivered to the User in a variety of formats such as audio, video, textual, graphical, and/or visual formats (“IQL Data Delivery”). For example, if the User selected the DDI located within an IQL under the “Sales Rep” Job Interview Heading and the Interview Question represented in such IQL was, “Are you willing to relocate?”, this question would be played back for the User to review (assuming the question was input to the system in an audio format or was converted to audio format by the system). See FIGS. 9 and 10 discussed below.

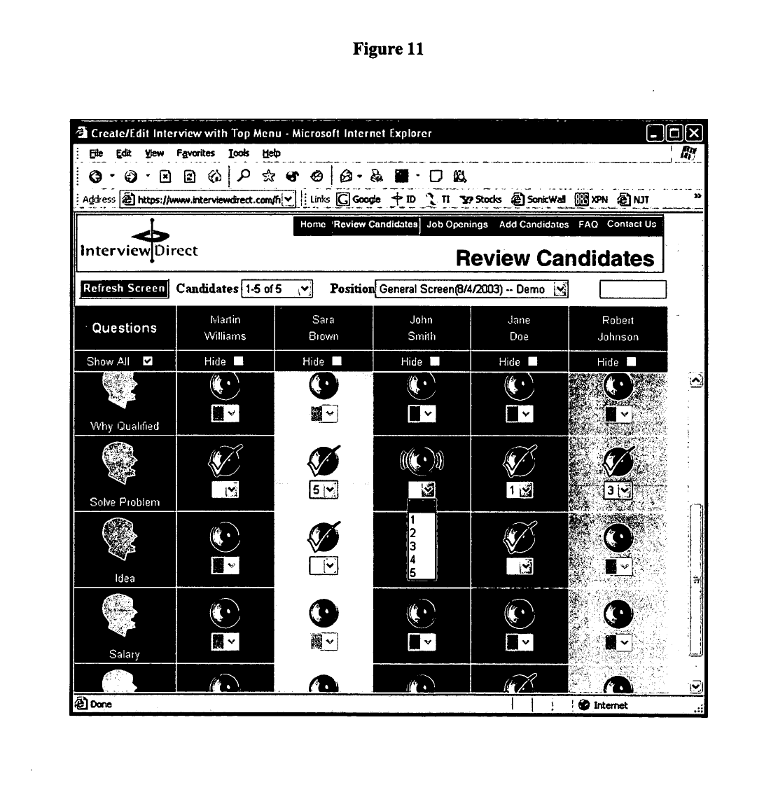

The User may input information into the system that the User designates to be associated with any given RDR contained within any ARL (“RDR User Input”) and such RDR User Input may be displayed or accessible from within, the ARL containing such RDR. For example, if an ARL contains an RDR of the word “yes” in textual format, the User could input to the system the number 2 for the system to associate with such RDR and the numeral 2 would then be displayed within the ARL containing such RDR. This would provide the User with a process for ranking the responses of job applicants represented in each ARL and have such rankings incorporated as part of the display grid, as shown in FIGS. 10-11.

The User may input information into the system that the User designates to be associated with any given IQR contained within any IQL (“IQR User Input”); and such IQR User Input may be displayed or accessible from within, the IQL containing such IQR. For example, if an IQL contains an IQR of the words “recent accomplishments” in textual format, the User could input to the system the number 4 for the system to associate with such IQR and the numeral 4 would then be displayed within the IQL containing such IQR. This could provide the User with a process for ranking the Interview Questions represented in each IQL and have such rankings incorporated as part of the display grid.

The User may input information into the system that the User designates to be associated with any given ARL Data Delivery (“ARL Data Delivery User Input”) and such ARL Data Delivery User Input may be displayed or accessible from within, the ARL containing the DDI activating such ARL Data Delivery. For example, if the User activates ARL Data Delivery upon selecting a DDI located within an ARL and the following audio response is delivered to the User “I specialize in patent litigation”, the User could input to the system the number 1 for the system to associate with such ARL Data Delivery and the numeral 1 would then be displayed within the ARL containing the DDI associated with such ARL Data Delivery. This could provide the User with a process for ranking the responses of job applicants accessed from within each ARL via ARL Data Delivery and have such rankings incorporated as part of the display grid, as shown in FIG. 11.

The User may input information into the system that the User designates to be associated with any given IQL Data Delivery (“IQL Data Delivery User Input”); and such IQL Data Delivery User Input may be displayed or accessible from within, the IQL containing the DDI activating such IQL Data Delivery. For example, if the User activates IQL Data Delivery upon selecting a DDI located within an IQL and the following question in audio format is delivered to the User, “Do you enjoy patent litigation?”, the User could input to the system the number 5 for the system to associate with such IQL Data Delivery and the numeral 5 would then be displayed within the IQL containing the DDI associated with such IQL Data Delivery. This could provide the User with a process for ranking the Interview Questions accessed from within each IQL via IQL Data Delivery and have such rankings incorporated as part of the display grid.

The present invention thus enables the User to: (i) eliminate the time spent completing interviews with job applicants who quickly reveal they are not qualified; (ii) reduce the time requirements for job applicant screening; (iii) make faster and better hiring decisions by comparing job applicants on the basis of uniform, job-related criteria; (iv) eliminate the time-consuming work required to coordinate interviews; (v) screen more job applicants faster; (vi) save money by reducing or eliminating the use of staffing agencies; (vii) reduce legal exposure by ensuring questions asked are first reviewed by the user's legal counsel; and (viii) screen job applicants that require bilingual skills more efficiently.

The system optionally enables the user to select or preprogram the format of the data presented by the system. The system optionally provides a process whereby data conversion tools are automatically utilized to convert data input by the user and/or collected from each candidate, to the format selected or preprogrammed by the User. Use of such data conversion tools could include application of speech to text conversion technology, text to speech conversion technology, and text to graphic conversion technology.

The system optionally enables the User to generate reports based on preprogrammed and/or user predefined criteria as applied to the data collected by the system from job applicants and/or data input to the system by the User. Such report generation may include synthesis and analysis of: (i) the responses of job applicants collected by the system; (ii) the Job Interviews and Interview Questions deployed by the system; (iii) the RDR User Input, IQR User Input, ARL Data Delivery User Input, and IQL Data Delivery User Input; (iv) the number of job applicants hired by the User that were evaluated using the system; and (v) the number of job applicants rejected by the User that were evaluated using the system.

The system may also be configured so that different User's have different levels of access to data maintained by the system. For example, only certain User's would be granted access by the system to certain Job Interviews, Interview Questions and only the responses of certain job applicants. Conversely, certain User's could be granted access by the system to all Job Interviews, Interview Questions and the responses of all job applicants.

Referring now to the drawings, FIG. 1 depicts a graphical user-interface for entering information about a position that will be the subject of automated interviews, in accordance with the present invention. The graphical user interface in FIG. 1 enables Users to enter a new job position and associated job description into the system, associate interview questions with the job, and select the deadline date that job applicants must respond to the automated interview. A “Job Position” is a designation associated by the system directly or indirectly with: (a) a Job title in text form (“Job Title”); (b) a description of an employment opportunity in text form (“Job Description”); (c) certain identification information associated with those Respondents (“Respondents' Contact Information”) selected by the User to be invited to take an Automated Interview selected by the User; (d) one or more unique identifiers assigned to Respondents by the system or the User (“PIN”); (e) an Automated Interview; (f) an Interview Deadline Date; and (g) the telephonic responses of Respondents (both verbal and keyed). In order to operate the interface shown in FIG. 1:

1. The User enters a Job Title in the “Job Title” field.

2. The User selects from a drop down menu in the “Job Interview” field, an Automated Interview from a selection of Automated Interviews provided to or created by the User to be associated with said Job Title.

3. The User enters a description of the employment opportunity into the “Job Description” field.

4. The User selects a date after which Respondents will no longer be able to submit responses to the Automated Interview selected by the User (“Interview Deadline Date”).

FIG. 2 depicts a graphical user-interface for entering information about candidates that will be interviewed, in accordance with the present invention. The graphical user interface shown in FIG. 2 enables Users to select an existing job position and enter job applicants whom the Users select to be interviewed by the automated job applicant interviewing system. In order to operate the interface shown in FIG. 2:

1. The User selects a Job Position from a drop down menu in the “Existing Job Positions” field.

2. The User enters Respondents' Contact Information for those Respondents that the User wants invited by the system to take the Automated Interview associated with such Job Position (“Selected Respondents”). The system may also be configured for the automated input for such Respondents' Contact Information from other data management or storage systems maintained by User.

In one embodiment, upon input to the system via the interfaces described in FIGS. 1 and 2 above, for such Selected Respondents the system will automatically:

1. Generate and assign a unique PIN for each of the Selected Respondents

2. Send an E-mail to each Selected Respondent using the e-mail address input by the User for each such Selected Respondent that includes the following information:
a. an invitation to provide information via an automated telephonic interview;
b. a telephone number to access the system in order to take the Automated Interview associated with such Job Position;
c. the PIN;
d. the Job Title;
e. the Job Description;
f. the Interview Deadline Date;
g. the Identity of the User; and
h. such other text and graphical information as the User prescribes.

3. Send a copy of each such E-mail to the User

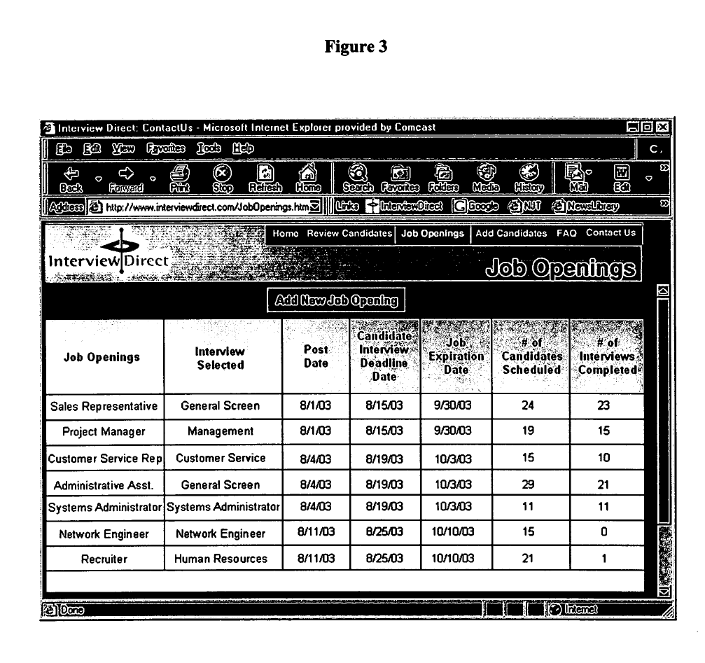

FIG. 3 depicts a graphical user-interface for displaying summary information about positions that are the subject of automated interviews, in accordance with the present invention. For each Job Opening, the interface of FIG. 3 displays: the following associated User Input data: 1) Job Position Creation (Post Date); Automated Interview (Interview Selected); and 3) Candidate Interview Deadline Date; and the following system generated data: 1) the number of candidates input by the user to take the Automated Interview (Number of Candidates Scheduled); 2) the number of candidates who have completed and submitted responses to an Automated Interview (Number of Interviews Completed); and 3) the date the Job Position can no longer be accessed by the User (Job Expiration Date).

FIG. 4 depicts a graphical user-interface for assembling a set of stored interview questions for a particular position that is the subject of automated interviews, in accordance with the present invention. The graphical user interface of FIG. 4 enables Users to build new automated interviews or modify existing automated interviews by dragging questions from the Question Bank area and dropping them into the Interview Assembly Area. Upon initial access of the interface of FIG. 4, a New Interview could be selected in the drop-down at the top of the page, all of the question squares in the Interview Assembly Area are empty, as is the text in the drop-down/information boxes associated with each such question square, and the General tab in the Question Bank is active.

The page shown in FIG. 4 would now be set for the User to create a completely new interview. The drop-down at the top of the page could be used to select previously created interviews. Upon selection of a previously created interview, the system populates the question squares in the Interview Assembly Area with the questions associated with the interview as well as the associated time limit for verbal response questions and type of question for touch-tone response questions. The talking head icon represents a question requiring a verbal response, the telephone icon represents a question requiring a touch-tone response.

The Question Bank at the bottom of the page shown in FIG. 4 could be divided into question categories that could be accessed by clicking on the associated category tab.

Clicking on the Record New Question button optionally initiates a dialog box that asks the user to enter in the phone number where he can be reached. Once the user submits the phone number to the system, the system's IVR interface automatically calls that phone number. The User may then be instructed by the IVR system to record a new question. Touch-tone phone responses could enable the User to review the recorded question, erase the recorded question to start over, and submit the recorded question to the system. Once the recorded question is submitted to the system the Select Question Attribute page (see FIG. 5 discussed below) can be automatically initiated. Once the User submits the associated question attributes, the User may returned to the Create/Edit Interview page, where the appropriate question icon along with its associated name appears automatically in the Custom Recorded section of the Question Bank, which becomes the active tab on the page. The user could then manipulate that question in the same way as any other question in the Question Bank.

To create a virtual interview, a User drags a question from the Question Bank to the desired question number in the Interview Assembly Area. If the question requires a touch-tone response, the type of touch-tone response (yes/no, multiple choice, numeric, etc.) is displayed in the grayed-out information box above the question box. If the question requires a verbal response, the default response time-limit, for instance, 30 seconds, appears in the drop-down above the question box. The user then selects a different time-limit to associate with each question requiring a verbal response. The order of questions may be changed simply by dragging the questions already in the Question Bank to alternate question numbers in the bank.

Clicking the Delete Interview button deletes the virtual interview that is selected in the drop-down at the top of the page. Clicking the Save Interview button permanently saves the virtual interview. If it is a new interview, a dialog box may open asking the User to select a name to call the virtual interview.

The graphical user interface shown in FIG. 4 may be used to build a plurality of different sequences of interview questions for conducting automated interviews of candidates, wherein each of the different sequences of interview questions corresponds to a different position. The graphical user interface streamlines the building of interview question sequences by facilitating the reuse of interview questions in multiple interview question sequences. Thus, once an interview question is recorded and stored in the database, the same interview question can be used to build interview question sequences for multiple different positions without re-recording of the question by simply dragging and dropping the icon representing the question from the Question Bank into the Interview Assembly Area during the building of the question sequences corresponding to the different positions.

FIG. 5 depicts a graphical user-interface for assigning attributes to interview questions, in accordance with the present invention. The interface in FIG. 5 enables Users to associate attributes with a newly recorded question. This page may be selected when the user completes recording a new question and hangs up the telephone. Clicking on the speaker icon at the top of the page enables the user to hear the question he or she just recorded. Before submitting the question to be saved, the User completes a short question description which could be used as the question file name. The User must also select whether or not the newly recorded question requires a verbal response or a touch-tone response. The Type of Touch-Tone Response drop-down selection could be disabled unless the Touch-Tone response radio button is selected. The default Type of Touch-Tone Response could be Yes/No, where the number 1 represents a yes response, and the number 2, a no response. Other valid selections, could include:

Multiple Choice—where the number 1 represents the first choice, and the number n represents the nth choice, where no more than 9 choices are allowed. If the User selects this option, the Valid Choices drop-down is enabled with valid selections ranging from 2 through 9.

Numeric—if this option is selected, the Number of Digits drop-down could be enabled with valid selections ranging from 1 through 7, enabling responses to range from zero through 9, or zero through 99, or zero through 999, etc., up to zero through 999,999,999.

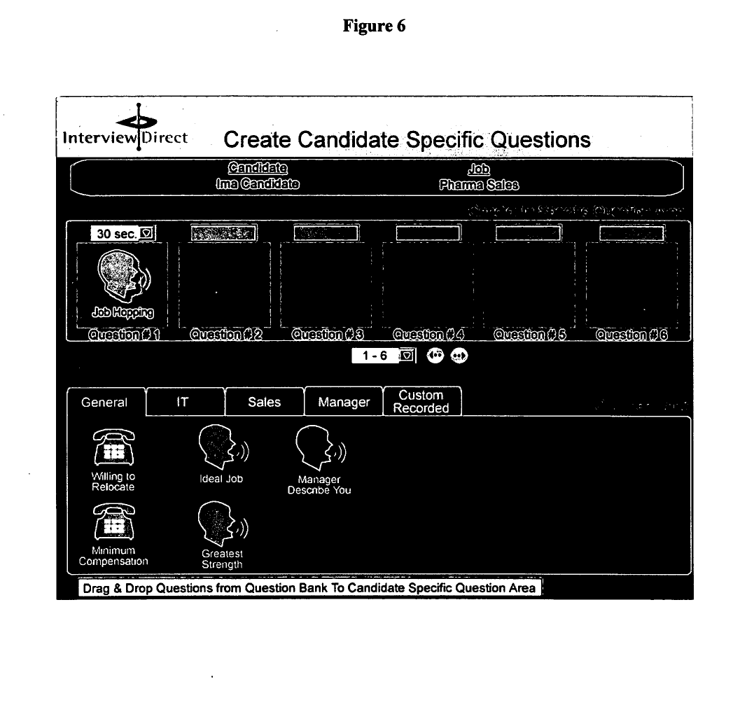

Clicking on the Submit New Question button saves the question and associated question attributes in the database and then returns the User to either the Create/Edit Interview page (FIG. 4) or the Create Candidate Specific Questions page (FIG. 6), depending on where the User initiated the Record New Question function. The appropriate question icon along with its associated name appears automatically in the Custom Recorded section of the Question Bank, which may then become the active tab on the page.

FIG. 6 depicts a graphical user-interface for assigning candidate specific questions to a candidate, in accordance with the present invention. The graphical user interface of FIG. 6 enables Users to add candidate specific questions to automated interviews by dragging questions from the Question Bank area and dropping them into the Candidate Specific Assembly Area. From a User functionality standpoint, operation of this screen is substantially the same as the Create/Edit Interviews Screen (FIG. 4).

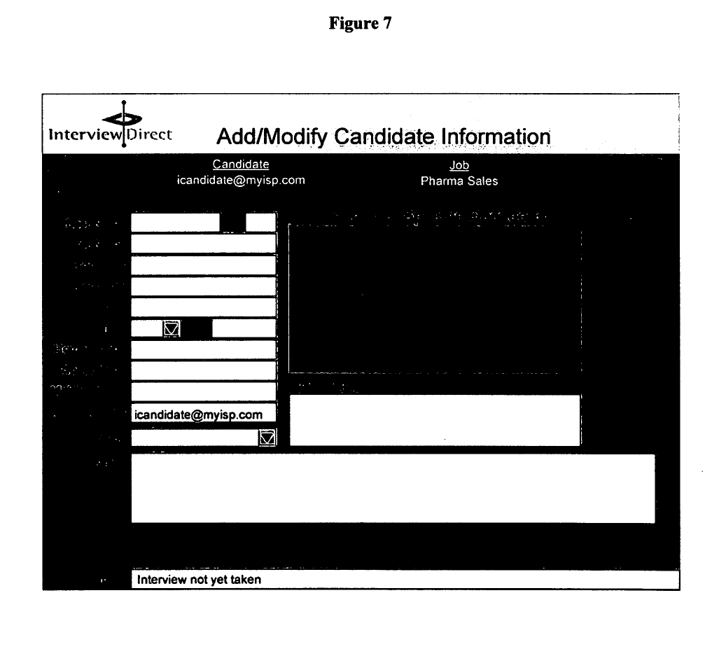

FIG. 7 depicts a graphical user-interface for adding/modifying candidate information, in accordance with the present invention. The graphical user interface of FIG. 7 enables the User to add or modify supplemental information about the job applicant (beyond the information entered through the Add Candidates screen (FIG. 2)).

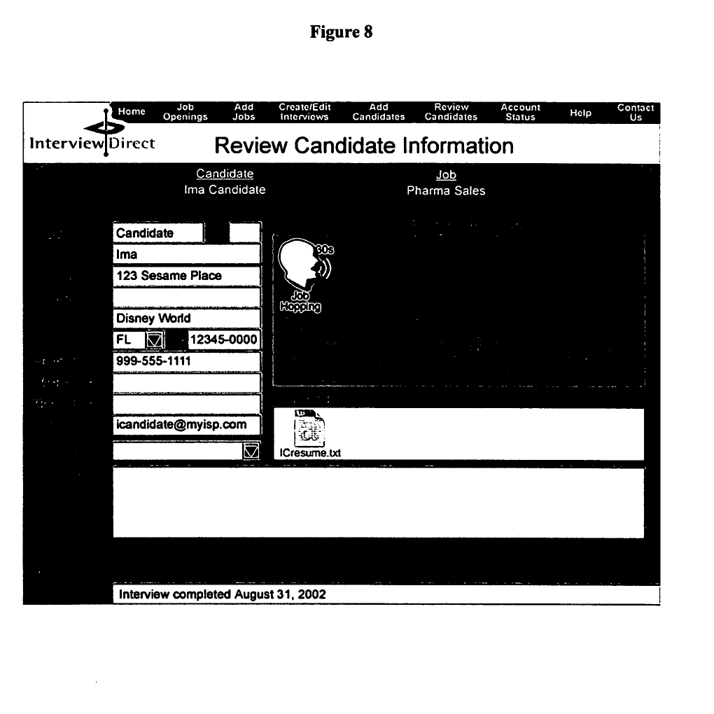

FIG. 8 depicts a graphical user-interface for reviewing candidate information, in accordance with the present invention. The graphical user interface of FIG. 8 enables the User to review information about the job applicant. This interface may include a link to the job applicant's resume, which could be displayed by clicking on such link, and provide for visual display of the resume while the user reviews the job applicant's interview responses.

FIG. 9 depicts a graphical user-interface for selectively reviewing verbal responses of candidates, in accordance with the present invention. This graphical user interface enables Users to review job applicant responses. The drop-down in the top center of the page enables Users to select candidates associated with a specific job opening. Clicking on the name heading optionally takes the User to the Review Candidate Information page (FIG. 8). In the case of interview questions requiring a verbal (or spoken, narrative) response from a candidate, clicking on a question icon invokes a streaming audio function that plays the question through the User's computer sound card. Clicking on a speaker icon in the candidate response area likewise invokes a streaming audio function that plays the candidate's audio response to the question. Clicking on a video icon (not shown) that may optionally be positioned in the candidate response area invokes a streaming audio/video function that plays the candidate's audio/video response in window 900. If the candidate's response was strictly audio, in place of the video icon an empty box with either the text, Video Not Available, inside, or a No-Video or similar icon may appear. The drop-downs under windows 900 enable the User to rate each candidate's response.

FIG. 10 depicts a further example of the graphical user-interface shown in FIG. 9. The example illustrates how the graphical user interface enables Users to review general job applicant responses, as well as candidate specific job applicant responses. This page represents the last in the series of job applicant questions/responses. The last question in any virtual interview could be one asking the candidate to clarify any of his previous responses and to ask any questions that he would like the hiring/HR manager to answer. The talking head icon labeled Candidate Questions and Comments represents this question which could be automatically appended to any automated interview. The question icons labeled Job Hopping represent a candidate specific question. Such icons may be color coded. The candidates whose automated interviews contain these questions could display the standard response icons. The candidates whose virtual interviews do not contain these questions could display the No-Question icon.

Among other things, the interface shown in FIGS. 9 and 10 allows the User to select and sequentially play back each candidate's recorded verbal response to a given Question, i.e., “What is your desired salary?” without playing back in between any verbal responses of the candidates to other Questions from the Interview. By allowing the User to juxtapose in time verbal responses from multiple candidates to the same Question, the present invention facilitates and streamlines the efficient and effective comparison of candidate responses received during the automated interviewing process.

FIG. 12 illustrates a system for implementing the functionality illustrated in FIGS. 1-11. In the system shown, one or more User(s) (at the Client Companies) access web server 1110 over the Internet (or other network). Web server 1110 supports the graphical user interfaces described above in connection with FIGS. 1-11. Web server 1110 is coupled via LAN 1150 to IVR servers 1140, which communicate with the interview candidates over telephone lines 1160 to perform the automated interviews described above. SQL database 1130 is coupled to LAN 1150 and stores various information about the automated interview process, including the data illustrated in FIGS. 1-11 above. Secure media server 1120 is also coupled to LAN 1150, and is used for storing audio questions and responses in connection with the automated interview process. It will be understood by those skilled in the art that various other hardware configurations could be used to implement the functionality of the present invention, and the particular configuration shown in FIG. 12 should not be deemed to limit the scope of the present invention.

Finally, it will be appreciated by those skilled in the art that changes could be made to the embodiments described above without departing from the broad inventive concept thereof. It is understood, therefore, that this invention is not limited to the particular embodiments disclosed, but is intended to cover modifications within the spirit and scope of the present invention as defined in the appended claims.

1. A computer-implemented method for interviewing at least first and second candidates and comparing candidate responses to interview questions, comprising:
(a) storing a plurality of interview questions for cueing the candidates, wherein the plurality of interview questions includes at least first and second questions;
(b) automatically interviewing the first candidate by:
(i) using an interactive voice response unit to sequentially prompt the first candidate with each of the plurality of stored interview questions; and
(ii) storing a verbal response of the first candidate to each of the interview questions in a database;
(iii) wherein step (b)(ii) includes storing a verbal response of the first candidate to the first interview question in the database and storing a verbal response of the first candidate to the second question in the database;
(c) automatically interviewing the second candidate by:
(i) using the interactive voice response unit to sequentially prompt the second candidate with each of the plurality of stored interview questions; and
(ii) storing a verbal response of the second candidate to each of the interview questions in the database;
(iii) wherein step (c)(ii) includes storing a verbal response of the second candidate to the first interview question in the database and storing a verbal response of the second candidate to the second question in the database;
wherein the verbal responses stored in steps (b) and (c) comprises audible narrative candidate responses;
(d) after steps (b) and (c), selecting, from the database, the stored verbal response of the first candidate to the first interview question and the stored verbal response of the second candidate to the first interview question; and
(e) comparing the stored verbal response of the first candidate to the first interview question to the stored verbal response of the second candidate to the first interview question;
wherein the comparing in step (e) includes sequentially playing the stored verbal response of the first candidate to the first interview question and the stored verbal response of the second candidate to the first interview question without playing any other stored interview question response between the playing of the stored verbal response of the first candidate to the first interview question and the stored verbal response of the second candidate to the first interview question.

2. The method of claim 1, wherein the stored verbal response of the first candidate to the first interview question and the stored verbal response of the second candidate to the first interview question are selected for review in step (d) using a graphical user interface.

3. The method of claim 2, wherein the graphical user interface contains first, second, third and fourth response areas, wherein the first response area corresponds to the stored verbal response of the first candidate to the first interview question, the second response area corresponds to the stored verbal response of the first candidate to the second interview question, the third response area corresponds to the stored verbal response of the second candidate to the first interview question, and the fourth response area corresponds to the stored verbal response of the second candidate to the second interview question.

4. The method of claim 3, wherein the first, second, third and fourth response areas are arranged in a grid pattern.

5. The method of claim 1, further comprising:
(f) after steps (b) and (c), selecting, from the database, the stored verbal response of the first candidate to the second interview question and the stored verbal response of the second candidate to the second interview question; and
(g) comparing the stored verbal response of the first candidate to the second interview question to the stored verbal response of the second candidate to the second interview question;
wherein the comparing in step (g) includes sequentially playing the stored verbal response of the first candidate to the second interview question and the stored verbal response of the second candidate to the second interview question without playing any other stored interview question response between the playing of the stored verbal response of the first candidate to the second interview question and the stored verbal response of the second candidate to the second interview question.

6. The method of claim 1, wherein the first and second candidates each correspond to a job applicant.

7. The method of claim 1, wherein the first and second candidates each correspond to a college applicant.

8. A computer-implemented system for interviewing at least first and second candidates and comparing candidate responses to interview questions, comprising:
(a) a database that stores a plurality of interview questions for cueing the candidates, wherein the plurality of interview questions includes at least first and second questions;
(b) an interactive voice response unit that:
(i) automatically interviews the first candidate by:
(a) sequentially prompting the first candidate with each of the plurality of stored interview questions; and
(b) storing a verbal response of the first candidate to the first interview question in the database and storing a verbal response of the first candidate to the second question in the database; and
(ii) automatically interviews the second candidate by:
(a) sequentially prompting the second candidate with each of the plurality of stored interview questions; and
(b) storing a verbal response of the second candidate to the first interview question in the database and storing a verbal response of the second candidate to the second question in the database;
wherein each of the verbal response comprises an audible narrative candidate response;
(d) an interface, operable after completion of the automatic interviewing of the first and second candidates by the interactive voice response unit, for selecting, from the database, the stored verbal response of the first candidate to the first interview question and the stored verbal response of the second candidate to the first interview question; and
(e) a processor, responsive to the interface, that sequentially plays the stored verbal response of the first candidate to the first interview question and the stored verbal response of the second candidate to the first interview question without playing any other stored interview question response between the playing of the stored verbal response of the first candidate to the first interview question and the stored verbal response of the second candidate to the first interview question.

9. A computer-implemented method for generating a plurality of different sequences of interview questions for conducting automated interviews of candidates, wherein each of the different sequences of interview questions corresponds to one or more of a plurality of different positions associated with the automated interviews, comprising:
(a) storing a plurality of different interview questions in a database;
(b) providing a graphical-user interface, coupled to the database, that displays a plurality of labels each of which represents one of the different interview questions, the graphical user-interface also including an assembly area for assembling each of the sequences of interview questions, wherein the graphical user interface simultaneously displays the plurality of labels and the assembly area;
(c) assembling a first sequence of interview questions corresponding to a first position by associating a first plurality of said labels with the assembly area and using the graphical user interface to associate the first sequence of questions with the first position;
(d) after step (c), assembling at least a second sequence of interview questions corresponding to a second position by associating a second plurality of said labels with the assembly area and using the graphical user interface to associate the second sequence of questions with the second position; wherein the first sequence of questions is different from the second sequence of questions; and wherein at least one label representing a common question is associated with the assembly area during assembly of the first sequence and during assembly of the second sequence; and
(e) using an interactive voice response unit, coupled to the database, to automatically interview candidates for the first position using the first sequence of interview questions assembled in step (c) and to automatically interview candidates for the second position using the second sequence of interview questions assembled in step (d).

10. The method of claim 9, wherein the plurality of labels comprises a plurality of icons each of which represents one of the different interview questions and step (b) comprises assembling the first sequence of interview questions corresponding to the first position by dragging and dropping a first plurality of said icons into the assembly area; and wherein step (c) comprises assembling the second sequence of interview questions corresponding to the second position by dragging and dropping a second plurality of said icons into the assembly area.

11. The method of claim 10, wherein each of the icons corresponds to a narrative interview question stored in an audible format in the database.

12. The method of claim 9, further comprising adding an interview question customized for a specific candidate for the first position to the first sequence of interview questions prior to automatically interviewing the specific candidate for the first position.

13. The method of claim 9, further comprising using the graphical user interface to associate one or more attributes with an interview question stored in the database.

14. A system for generating a plurality of different sequences of interview questions for conducting automated interviews of candidates, wherein each of the different sequences of interview questions corresponds to one or more of a plurality of different positions associated with the automated interviews, comprising:
(a) a database that stores a plurality of different interview questions;
(b) a graphical-user interface, coupled to the database, that displays a plurality of labels each of which represents one of the different interview questions, the graphical user-interface also including an assembly area for assembling each of the sequences of interview questions, wherein the graphical user interface simultaneously displays the plurality of labels and the assembly area;
(c) the graphical user interface including functionality operable by a user for assembling a first sequence of interview questions corresponding to a first position by associating a first plurality of said labels with the assembly area and functionality operable by the user for associating the first sequence of questions with the first position;
(d) the graphical user interface including functionality operable by the user for assembling at least a second sequence of interview questions corresponding to a second position by associating a second plurality of said labels with the assembly area and functionality operable by the user for associating the second sequence of questions with the second position; wherein the first sequence of questions is different from the second sequence of questions; and wherein the graphical user interface includes functionality operable by the user for associating at least one label representing a common question with the assembly area during assembly of the first sequence and for associating the at least one label representing the common question with the assembly area during assembly of the second sequence; and
(e) an interactive voice response unit, coupled to the database, that automatically interviews candidates for the first position using the first sequence of interview questions assembled using the graphical user interface and automatically interviews candidates for the second position using the second sequence of interview questions assembled using the graphical user interface.

15. A computer-implemented method for generating at least one interview question for conducting an automated interview of a candidate for a position, comprising:
(a) submitting, by a user, a request to a server to create an interview question;
(b) after step (a), using the server to prompt the user to input a label to be assigned to the interview question and receiving the label from the user;
(c) during a telephone call established after step (b) between the user and an interactive voice response unit coupled to the server, prompting the user, with the interactive voice response unit, to speak the interview question;
(d) recording the interview question spoken by the user during the telephone call and storing the recorded interview question in a database associated with the server; and
(e) conducting an automated interview of the candidate for the position by at least playing the stored question for the candidate during the automated interview and automatically recording and storing a response of the candidate to the stored interview question.

16. The method of claim 15, wherein step (b) further comprises: after step (a), using the server to prompt the user to input a telephone number associated with the user, receiving the telephone number from the user, and automatically initiating the telephone call to the user using the telephone number received from the user.

17. The method of claim 15, wherein step (b) further comprises: prompting the user to input a time duration time associated with a response to the interview question.

18. The method of claim 15, wherein step (d) further comprises: providing the user with an option to listen to the recorded interview question, and an option to rerecord the interview question.

19. A system for generating at least one interview question for conducting an automated interview of a candidate for a position, comprising:
(a) a server that receives a request submitted by a user to create an interview question, prompts the user to input a label to be assigned to the interview question and receives the label from the user;
(b) at least one interactive voice response unit, coupled to the server, that conducts a telephone call, established after receipt of the request, with the user, wherein the at least one interactive voice response unit prompts the user to speak the interview question during the telephone call;
(d) a database, coupled to the server and the at least one interactive voice response unit, that stores a recording of the interview question spoken by the user during the telephone call; and
(e) wherein the at least one interactive voice response unit conducts an automated interview of the candidate for the position by at least playing the stored question for the candidate during the automated interview.
